(19) United States
(12) Patent Application Publication
(10) Pub. No.: US 2024/0221883 A1
(43) Pub. Date: Jul. 4, 2024

(54) PREDICTION APPARATUS, LEARNING APPARATUS, PREDICTION METHOD, LEARNING METHOD AND PROGRAM

(71) Applicant: NIPPON TELEGRAPH AND TELEPHONE CORPORATION, Tokyo (JP)

(72) Inventors: Tomu TOMINAGA, Tokyo (JP); Shuhei YAMAMOTO, Tokyo (JP); Takeshi KURASHIMA, Tokyo (JP); Hiroyuki TODA, Tokyo (JP)

(21) Appl. No.: 18/557,151

(22) PCT Filed: Jun. 2, 2021
(86) PCT No.: PCT/JP2021/021055

§ 371 Date: Oct. 25, 2023

(51) Int. Cl. G16H 20/00

(52) U.S. Cl. CPC G16H 20/00

(57) ABSTRACT

Provided is a prediction apparatus for predicting a health-oriented behavior tendency of a prediction object, the prediction apparatus including: a health-oriented behavior estimation model storage unit that stores a health-oriented behavior estimation model for estimating a health-oriented behavior based on a relationship between a health-oriented behavior and human characteristics; a human characteristic scale calculation unit that calculates a plurality of human characteristic scales based on human characteristic data indicating human characteristics of a prediction object; and a health-oriented behavior prediction unit that predicts a health-oriented behavior tendency based on the plurality of calculated human characteristic scales by using the health-oriented behavior estimation model.

### TECHNICAL FIELD

The present invention relates to a prediction apparatus, a learning apparatus, a prediction method, a learning method, and a program.

### BACKGROUND ART

When individuals set goals to be achieved regarding their health, it is an important issue to understand a series of goal-oriented behaviors whereby the goals are achieved. In recent years, due to the widespread use of healthcare applications and fitness devices, it has become possible to observe, on a large scale, various logs regarding behavioral processes of people to become healthier. For example, in many weight management mobile applications, users first set a target weight and then record their weight over a certain period of time. In this case, from the recorded data, data on a series of goal-oriented behaviors in weight management, such as a user's initial weight (initial value), a target weight relative to the current value (target difficulty level), actual weight loss relative to the current value (degree of execution), and actual weight loss relative to the target weight (degree of achievement), can be measured.

Based on such logs, technology has been developed to analyze a relationship between a health-oriented behavior and human characteristics in order to understand behavioral patterns that lead to a healthier state for users. Here, the term “human characteristics” refers to the inherent or static nature of a user at a certain point in time, and is mainly detected through self-report questionnaire surveys. As conventional technologies, there have been devised methods for estimating health-oriented behaviors such as high target values in dieting (NPL 1) and actual weight loss (NPL 2) based on human characteristics such as gender, age, and body mass index (BMI).

### RELATED ART LITERATURE

### Non Patent Literature

Non Patent Literature 1: Mitchell Gordon, Tim Althoff, and Jure Leskovec. 2019. Goal-setting And Achievement In Activity Tracking Apps: A Case Study Of MyFitnessPal. In The World Wide Web Conference (WWW '19).
Non Patent Literature 2: Zhiwei Wang, Tyler Derr, Dawei Yin, and Jiliang Tang. 2017. Understanding and Predicting Weight Loss with Mobile Social Networking Data. In Proceedings of the 2017 ACM on Conference on Information and Knowledge Management (CIKM '17).

### SUMMARY OF INVENTION

### Technical Problem

However, the above-mentioned conventional technologies have focused on the process until the user achieves the goal, and cannot be evaluated from the viewpoint of how much the state reached by the achievement of the goal is retained in the user after that (degree of retention). Further, although basic information such as age, gender, and BMI is considered as human characteristics for predicting the health-oriented behavior of the user, other information such as demographic information, psychological characteristics, cognitive characteristics, health habits, and work productivity are not included, and sufficient prediction accuracy based on human characteristics has not been achieved.

An object of the disclosed technology is to improve prediction accuracy of health-oriented behavior tendencies.

### Solution to Problem

The disclosed technology is a prediction apparatus for predicting a health-oriented behavior tendency of a prediction object, the prediction apparatus including: a health-oriented behavior estimation model storage unit that stores a health-oriented behavior estimation model for estimating a health-oriented behavior based on a relationship between a health-oriented behavior and human characteristics; a human characteristic scale calculation unit that calculates a plurality of human characteristic scales based on human characteristic data indicating human characteristics of a prediction object; and a health-oriented behavior prediction unit that predicts a health-oriented behavior tendency based on the plurality of calculated human characteristic scales by using the health-oriented behavior estimation model.

### Advantageous Effects of Invention

The prediction accuracy of health-oriented behavior tendencies can be improved.

### BRIEF DESCRIPTION OF DRAWINGS

FIG. 1 is a functional configuration diagram of a learning apparatus.

FIG. 2 is a functional configuration diagram of a prediction apparatus.

FIG. 3 is a flowchart illustrating an example of a flow of learning processing.

FIG. 4 is a flowchart illustrating an example of a flow of prediction processing.

FIG. 5 is a diagram illustrating an example of health target data.

FIG. 6 is a diagram illustrating an example of health behavior data.

FIG. 7 is a diagram illustrating an example of preprocessed health behavior data.

FIG. 8 is a diagram illustrating an example of a calculation result of health-oriented behavior tendencies.

FIG. 9 is a diagram illustrating an example of human characteristic data.

FIG. 10 is a diagram illustrating an example of preprocessed human characteristic data.

FIG. 11 is a diagram illustrating an example of a human characteristic scale.

FIG. 12 is a diagram illustrating an example of a hardware configuration of a computer.

### DESCRIPTION OF EMBODIMENTS

Hereinafter, an embodiment of the present invention (the present embodiment) will be described with reference to the drawings. The embodiment described below is merely an example, and embodiments to which the present invention is applied are not limited to the following embodiment.

In the text of the body of the present specification, for convenience of description, a hat “\hat{ }” that should be placed above the head of the character is prefixed before the character. “\hat{ }r” is an example of this.

### Overview of Present Embodiment

A prediction apparatus according to the present embodiment estimates an initial value, a target difficulty level, a degree of execution, a degree of achievement, and a degree of retention indicated by a health-oriented behavior scale of a prediction object (person) on the basis of human characteristic data. The learning apparatus according to the present embodiment learns parameters of a health-oriented behavior estimation model used by the prediction apparatus on the basis of data of a learning object (person). Human characteristic data is data indicating human characteristics of a prediction object or the learning object, refers to the inherent or static nature of the prediction object or the learning object at a certain point in time, and is mainly data detected by a self-report questionnaire survey or the like.

### Example of Functional Configuration of Learning Apparatus

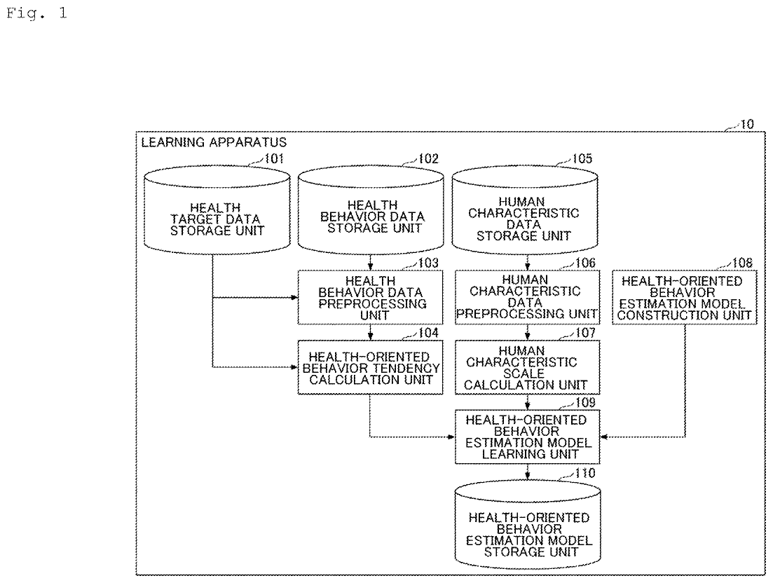

FIG. 1 is a functional configuration diagram of a learning apparatus. A learning apparatus 10 includes a health target data storage unit 101, a health behavior data storage unit 102, a health behavior data preprocessing unit 103, a health-oriented behavior tendency calculation unit 104, a human characteristic data storage unit 105, a human characteristic data preprocessing unit 106, a human characteristic scale calculation unit 107, a health-oriented behavior estimation model construction unit 108, a health-oriented behavior estimation model learning unit 109, and a health-oriented behavior estimation model storage unit 110.

The health target data storage unit 101 stores health target data in which a target value in health management of the learning object, a time when the target value is set (declaration time), and a period (target period) which the learning object has self-declared as a time required for achieving the target value are associated with a user ID. The user ID is an identifier for identifying the learning object.

The health behavior data storage unit 102 stores health behavior data in which a log of physical information (e.g., weight, body fat percentage, BMI, etc.) to be a target of health management of the learning object is associated with the user ID together with a recording time.

The human characteristic data storage unit 105 stores human characteristic data in which answers to questions in a self-report questionnaire survey or the like for capturing human characteristics of the learning object are associated with the user ID.

The health behavior data preprocessing unit 103 preprocesses the health behavior data. Specifically, the health behavior data preprocessing unit 103 determines a period to be analyzed on the basis of a target period recorded in the health target data, and extracts health behavior data corresponding to the determined period. The extracted health behavior data is defined as preprocessed health behavior data.

The health-oriented behavior tendency calculation unit 104 calculates a value indicating the health-oriented behavior tendency on the basis of the health target data and the preprocessed health behavior data.

The human characteristic data preprocessing unit 106 preprocesses the human characteristic data. Specifically, the human characteristic data preprocessing unit 106 converts the human characteristic data into a predetermined format in accordance with the nature of scales of answer values to question items of a questionnaire survey. The converted human characteristic data is defined as preprocessed human characteristic data.

The human characteristic scale calculation unit 107 calculates a plurality of human characteristic scales on the basis of the preprocessed human characteristic data.

The health-oriented behavior estimation model construction unit 108 constructs a health-oriented behavior estimation model. The health-oriented behavior estimation model is an estimation model for estimating the health-oriented behavior on the basis of a relationship between the health-oriented behavior and the human characteristics.

The health-oriented behavior estimation model learning unit 109 learns the health-oriented behavior estimation model on the basis of a value indicating the health-oriented behavior tendency calculated by the health-oriented behavior tendency calculation unit 104 and a specific human characteristic scale calculated by the human characteristic scale calculation unit 107, and outputs learned parameters of the health-oriented behavior estimation model.

The health-oriented behavior estimation model storage unit 110 stores the health-oriented behavior estimation model constructed by the health-oriented the parameters of the health-oriented behavior estimation model learned by the health-oriented behavior estimation model learning unit 109.

### Example of Functional Configuration of Prediction Apparatus

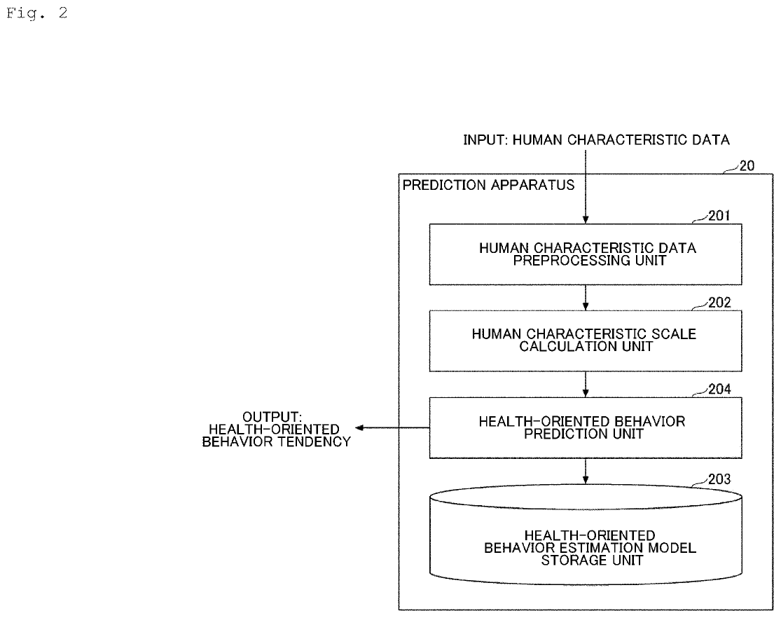

FIG. 2 is a functional configuration diagram of a prediction apparatus. A prediction apparatus 20 includes a human characteristic data preprocessing unit 201, a human characteristic scale calculation unit 202, a health-oriented behavior estimation model storage unit 203, and a health-oriented behavior prediction unit 204.

The human characteristic data preprocessing unit 201 applies preprocessing similar to that of the human characteristic data preprocessing unit 106 of the learning apparatus 10 to human characteristic data of a prediction object, and outputs preprocessed human characteristic data.

The human characteristic scale calculation unit 202 calculates a plurality of human characteristic scales by calculation similar to that of a human characteristic scale calculation unit 107 of the learning apparatus 10 on the basis of the preprocessed human characteristic data of the prediction object.

The health-oriented behavior estimation model storage unit 203 stores the health-oriented behavior estimation model constructed by the health-oriented the parameters of the health-oriented behavior estimation model learned by the health-oriented behavior estimation model learning unit 109, which are stored in the health-oriented behavior estimation model storage unit 110 of the learning apparatus 10.

The health-oriented behavior prediction unit 204 predicts a health-oriented behavior tendency based on the plurality of human characteristic scales calculated by the human characteristic scale calculation unit 202 by using the health-oriented behavior estimation model and the learned parameters of the health-oriented behavior estimation model, and outputs data indicating a prediction result.

### Operation Example of Learning Apparatus

Next, an operation example of the learning apparatus 10 will be described with reference to the drawings. The learning apparatus 10 starts learning processing in response to a user's operation or the like or periodically.

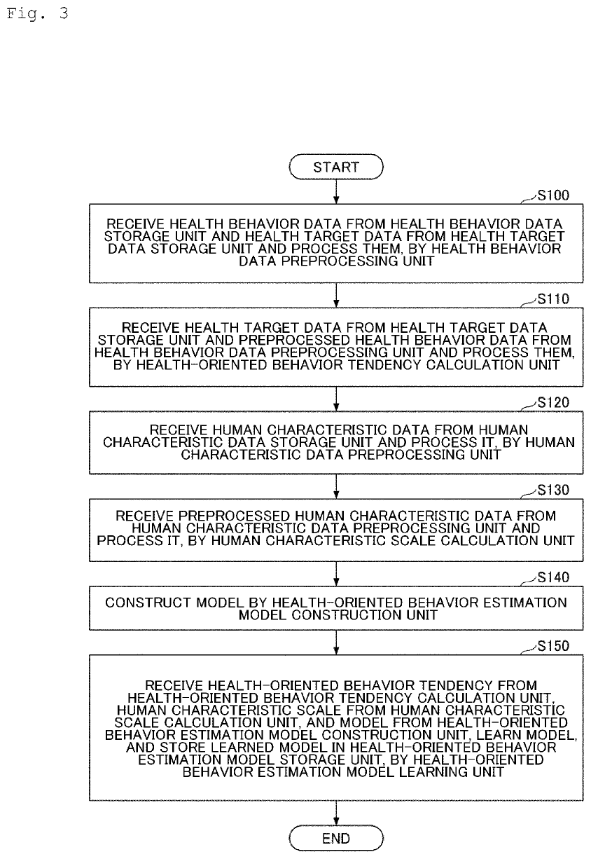

FIG. 3 is a flowchart illustrating an example of a flow of the learning processing. The health behavior data preprocessing unit 103 receives the health behavior data from the health behavior data storage unit 102 and the health target data from the health target data storage unit 101 and processes them (step S100). Specifically, the health behavior data preprocessing unit 103 determines a period to be analyzed on the basis of a target period recorded in the health target data, and extracts health behavior data corresponding to the determined period. The extracted health behavior data is defined as preprocessed health behavior data.

Next, the health-oriented behavior tendency calculation unit 104 receives the health target data from the health target data storage unit 101 and the preprocessed health behavior data from the health behavior data preprocessing unit 103 and processes them (step S110). Specifically, the health-oriented behavior tendency calculation unit 104 calculates a value indicating the health-oriented behavior tendency on the basis of the health target data and the preprocessed health behavior data.

Next, the human characteristic data preprocessing unit 106 receives the human characteristic data from the human characteristic data storage unit 105 and processes it (step S120). Specifically, the human characteristic data preprocessing unit 106 converts the human characteristic data into a predetermined format in accordance with the nature of the scales of the answer values to the question items of the questionnaire survey. The converted human characteristic data is defined as preprocessed human characteristic data.

Subsequently, the human characteristic scale calculation unit 107 receives the preprocessed human characteristic data from the human characteristic data preprocessing unit 106 and processes it (step S130). Specifically, the human characteristic scale calculation unit 107 calculates a specific human characteristic scale on the basis of the preprocessed human characteristic data.

Next, the health-oriented behavior estimation model construction unit 108 constructs a model (step S140). The model to be constructed is a health-oriented behavior estimation model for estimating the health-oriented behavior on the basis of the relationship between the health-oriented behavior and the human characteristics.

Next, the health-oriented behavior estimation model learning unit 109 receives the health-oriented behavior tendency from the health-oriented behavior tendency calculation unit 104, the human characteristic scale from the human characteristic scale calculation unit 107, and the model from the health-oriented behavior estimation model construction unit, learns the model, and stores the learned model in the health-oriented behavior estimation model storage unit (step S150). Specifically, the health-oriented behavior estimation model learning unit 109 learns the health-oriented behavior estimation model on the basis of a value indicating the health-oriented behavior tendency calculated by the health-oriented behavior tendency calculation unit 104 and the specific human characteristic scale calculated by the human characteristic scale calculation unit 107, and stores learned parameters of the health-oriented behavior estimation model in the health-oriented behavior estimation model storage unit 110.

### Operation Example of Prediction Apparatus

Next, an operation example of the prediction apparatus 20 will be described with reference to the drawings. The prediction apparatus 20 starts prediction processing in response to a user's operation or the like or periodically.

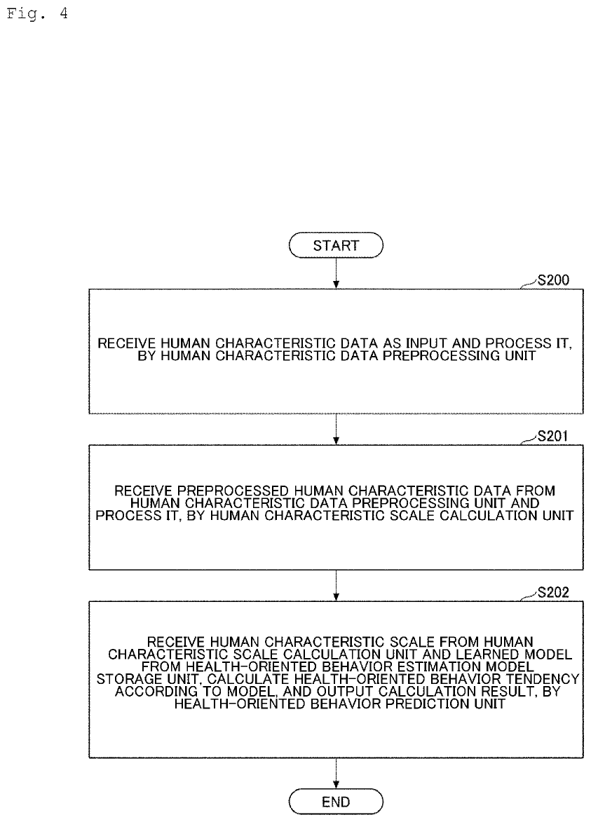

FIG. 4 is a flowchart illustrating an example of a flow of the prediction processing. The human characteristic data preprocessing unit 201 receives the human characteristic data as an input and processes it (step S200). Specifically, the human characteristic data preprocessing unit 201 converts the human characteristic data into a predetermined format in accordance with the nature of the scales of the answer values to the question items of the questionnaire survey, similarly to the process of step S120 of the learning processing by the learning apparatus 10. The converted human characteristic data is defined as preprocessed human characteristic data.

Next, the human characteristic scale calculation unit 107 receives the preprocessed human characteristic data from the human characteristic data preprocessing unit 106 and processes it (step S201). Specifically, the human characteristic scale calculation unit 107 calculates a specific human characteristic scale on the basis of the preprocessed human characteristic data, similarly to the process of step S130 of the learning processing by the learning apparatus 10.

Next, the health-oriented behavior prediction unit 204 receives the human characteristic scale from the human characteristic scale calculation unit 202 and the learned model from the health-oriented behavior estimation model storage unit 203, calculates a health-oriented behavior tendency according to the model, and outputs a calculation result (step S202). Specifically, the health-oriented behavior prediction unit 204 predicts the health-oriented behavior tendency based on the specific human characteristic scale calculated by the human characteristic scale calculation unit 202 by using the health-oriented behavior estimation model and the learned parameters of the health-oriented behavior estimation model, and outputs data indicating a prediction result.

### Format Examples of Various Types of Data According to Present Embodiment

Next, format examples of various types of data handled by the learning apparatus 10 and the prediction apparatus 20 according to the present embodiment will be described.

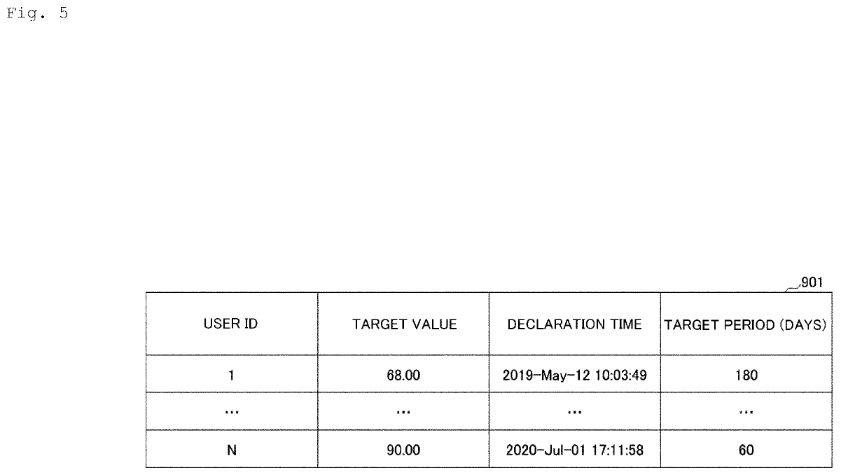

FIG. 5 is a diagram illustrating an example of the health target data. Health target data 901 includes, as items, a user ID, a target value, a declaration time, and a target period.

The value of the item “user ID” is an identifier for identifying the learning object. The value of the item “target value” is a target value in health management of the learning object. The value of the item “declaration time” is a time when the learning object sets the target value. The value of the item “target period” is a period in which the learning object self-declares as a time required for achieving the target value.

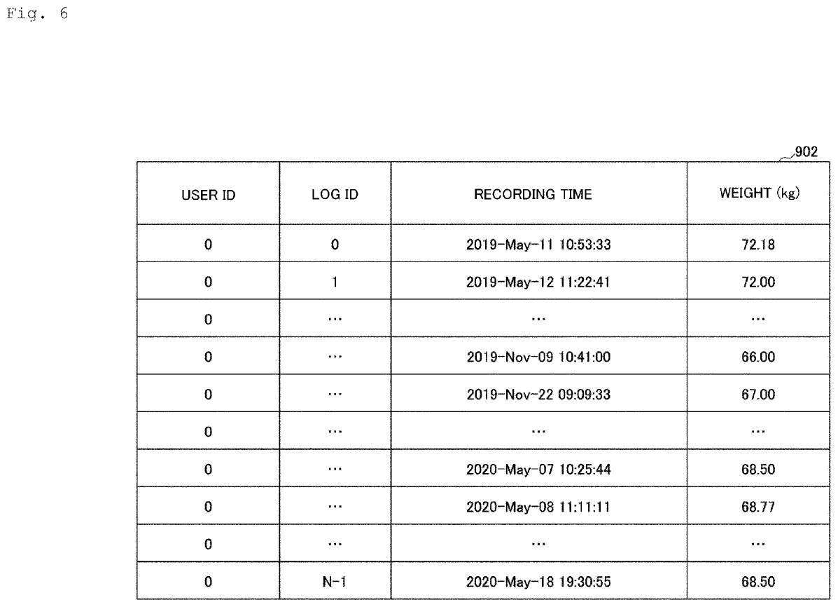

FIG. 6 is a diagram illustrating an example of the health behavior data. Health behavior data 902 includes, as items, a user ID, a log ID, a recording time, and a weight.

The value of the item “user ID” is an identifier for identifying the learning object. The value of the item “log ID” is an identifier for identifying a log. The value of the item “recording time” is a time when the log is recorded. The value of the item “weight” is a weight as an example of physical information to be a target of health management of the learning object.

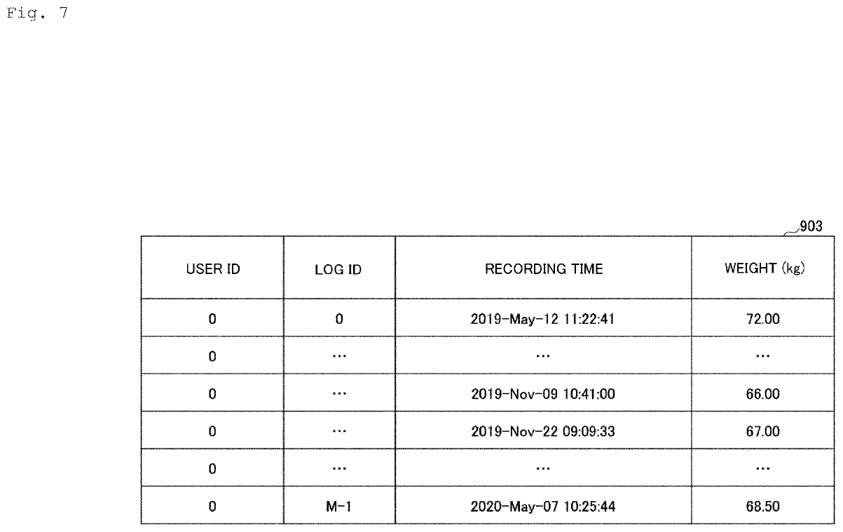

FIG. 7 is a diagram illustrating an example of the preprocessed health behavior data. Preprocessed health behavior data 903 includes, as items, a user ID, a log ID, a recording time, and a weight.

The value of the item “user ID” is an identifier for identifying the learning object. The value of the item “log ID” is an identifier for identifying the log. Since the value of the item “log ID” is newly reassigned in the preprocessing by the health behavior data preprocessing unit 103, it does not necessarily match the value of the item “log ID” of the health behavior data 902 illustrated in FIG. 6. The value of the item “recording time” is a time when the log is recorded. The value of the item “weight” is a weight as an example of physical information to be a target of health management of the learning object.

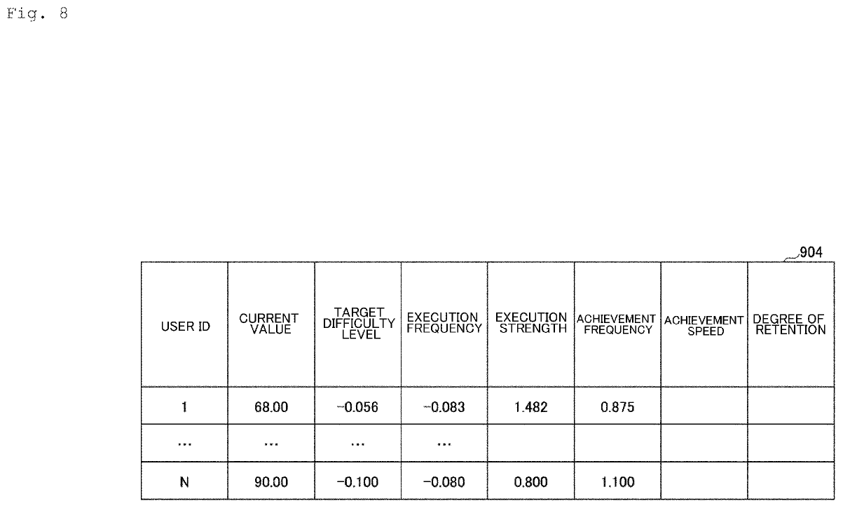

FIG. 8 is a diagram illustrating an example of a calculation result of health-oriented behavior tendencies. Each value of a calculation result 904 is a value indicating the health-oriented behavior tendency calculated by the health-oriented behavior tendency calculation unit 104. Specifically, the calculation result 904 is a value indicating a plurality of health-oriented behavior tendencies including a current value for each user ID, a target difficulty level, an execution frequency, an execution strength, an achievement frequency, an achievement speed, a degree of retention, and the like.

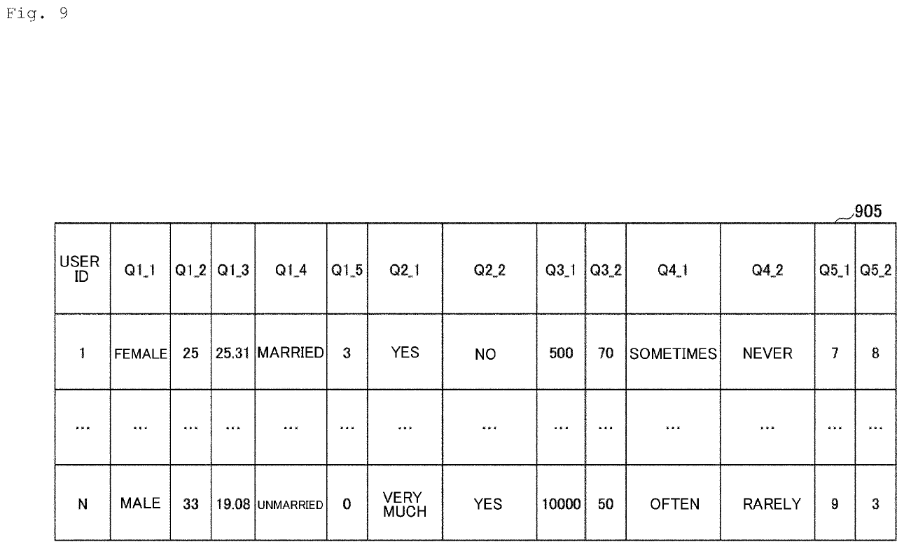

FIG. 9 is a diagram illustrating an example of the human characteristic data. Human characteristic data 905 is data in which an answer to a question from a self-report questionnaire survey or the like for capturing human characteristics of the prediction object or the learning object is associated with a user ID.

For example, the example of FIG. 9 is answers to the following questions.
Q1_1 to Q1_5: Questions about demographic information (Q1_1: Gender, Q1_2: Age, Q1_3: BMI, Q1_4: Marriage status, Q1_5: Number of housemates)
Q2_1 and Q2_2: Questions about psychological characteristics (Q2_1: “Do you like talking to people?”, Q2_2: “Are you not good at being with large groups of people?”/Answer options: Not at all, No, Neither, Yes, Very much)
Q3_1 and Q3_2: Questions about cognitive characteristics (Q3_1: “If there is a lottery with a 50% chance of winning 100,000 yen, up to how many yen would you pay to buy that lottery ticket?”, Q3_2: “At more than what percentage of probability of rain, would you take an umbrella?”)
Q4_1 and Q4_2: Questions about health habits (Q4_1: “Have you been unable to sleep at night lately?”, Q4_2: “Have you ever eaten fatty foods?”/Answer options: Never, Rarely, Sometimes, Often)
Q5_1 and Q5_2: Questions about work productivity (Q5_1: “Please rate your work performance over the past one year on a scale of 0 to 10”/Q5_2: “Please rate your work performance over the past four weeks on a scale of 0 to 10”)

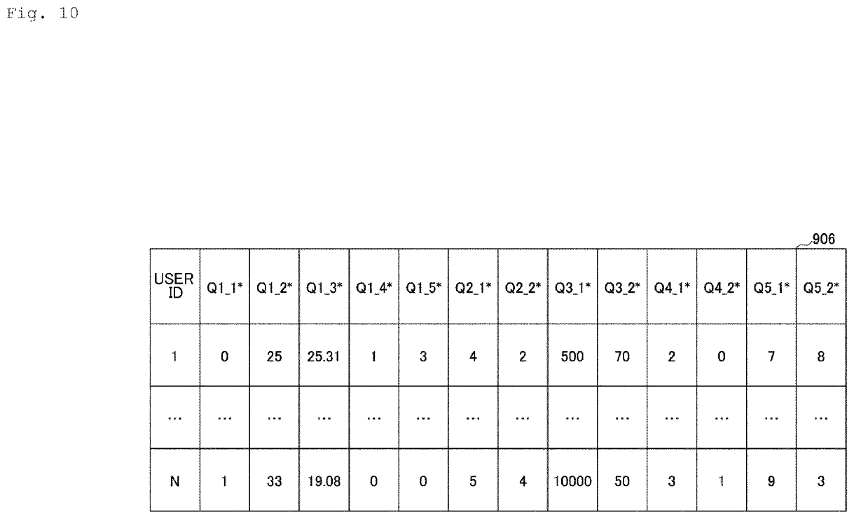

FIG. 10 is a diagram illustrating an example of the preprocessed human characteristic data. Preprocessed human characteristic data 906 is obtained by converting the human characteristic data 905 into a predetermined format.

For example, in the example of FIG. 10, the human characteristic data 905 illustrated in FIG. 9 is preprocessed by the human characteristic data preprocessing unit 106 in accordance with the following correspondence relationship.
Gender: (Q1_1*: Q1_1)=(0: Female) or (1: Male)
Age: Q1_2*=Q1_2
BMI: Q1_3*=Q1_3
Marriage status: (Q1_4*: Q1_4)=(0: Unmarried) or (1: Married)
Number of housemates: Q1_5*=Q1_5
Psychological characteristics:
(Q2_1*: Q2_1)=(1: Not at all), (2: No), (3: Neither), (4: Yes) or (5: Very much)
(Q2_2*: Q2_2)=(5: Not at all), (4: No), (3: Neither), (2: Yes) or (1: Very much)
Cognitive characteristics:
Q3*=Q3
Both Q3_1 and Q3_2 follow the above conversion.
Health habits: (Q4*: Q4)=(0: Never), (1: Rarely), (2: Sometimes), or (3: Often)
Both Q4_1 and Q4_2 follow the above conversion.
Work productivity: Q5*=Q5
Both Q5_1 and Q5_2 follow the above conversion.

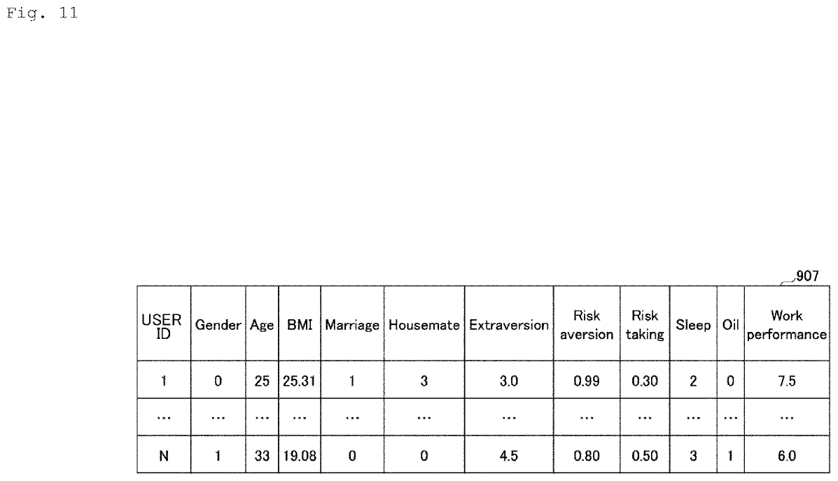

FIG. 11 is a diagram illustrating an example of the human characteristic scale. A calculation result 907 of the human characteristic scale includes various values indicating the human characteristic scale calculated by the human characteristic scale calculation unit 107. For example, the calculation result 907 illustrated in FIG. 11 is calculated by the following calculation based on the preprocessed human characteristic data 906 illustrated in FIG. 10.
Gender: Q1_1*
Age: Q1_2*
BMI: Q1_3*
Marriage: Q1_4*
Housemate: Q1_5+
Extraversion: (Q2_1*+Q2_2*)/2
Risk aversion: 1−Q3_1*/50000

The denominator of the second term in the above formula is the expected value of the reward obtained from this lottery.
Risk taking: 1−Q3_2*/100

The second term in the above formula is a value obtained by converting the answered probability of rain (%) into a probability value.
Sleep: Q4_1*
Oil: Q4_2*
Work Performance: (Q5_1*+Q5_2*)/2

### Details of Each Process According to Present Embodiment

Next, details of the learning processing and the prediction processing according to the present embodiment will be described. Hereinafter, when the i-th health behavior data log is yᵢ, the health behavior data vector is set to y=(y₀, . . . , Y_{N-1}) T, and the time when yᵢ is recorded is set to tᵢ. Also, the target value of the health target data is set to g, the declaration time is set to t_{g}, and the target period is set to s. Here, it is assumed that the health target data is constant unless updated.

When the health target data is updated during the target period or when the health target data is newly set after the end of the target period, the learning apparatus 10 executes the following process after updating the target value, the declaration time, and the target period.

The process by the health behavior data preprocessing unit 103 (step S100 of the learning processing illustrated in FIG. 3) will be described. The health behavior data preprocessing unit 103 extracts, from y, data recorded in a period from t_{g} to t_{g+s}. That is, a preprocessed health behavior data vector y* obtained by the health behavior data preprocessing unit 103 is calculated by the following Equation (1).

$$[ Math . 1 ]$$

$${y}^{*} = {( {y}_{j}^{*} \vert {t}_{i} ∈ [ {t}_{g} , {t}_{g} + s ] , j = i - {i}_{g} )}^{T} = {( {y}_{0}^{*} , … , {y}_{M - 1}^{*} )}^{T} \qquad ( 1 )$$

Here, i_{g} denotes the log ID of the health behavior data first recorded in tᵢΣ[t_{g}, t_{g+s}]. The time when the yᵢ* is recorded is denoted by tᵢ*.

Next, the process by the health-oriented behavior tendency calculation unit 104 (step S110 of the learning processing illustrated in FIG. 3) will be described. The health-oriented behavior tendency calculation unit 104 extracts a current value r^{(s)}, a target difficulty level r^{(d)}, a degree of execution r^{(p)}, a degree of achievement r^{(a)}, and a degree of retention r^{(f)} from the preprocessed health behavior data vector y* and the target value g.

Among these, the current value refers to the first recorded data in the preprocessed behavior data. The current value of a learning object u is calculated by the following Equation (2).

$$[ Math . 2 ]$$

$${r}^{( s )} = {y}_{0}^{*} \qquad ( 2 )$$

The target difficulty level is a value that expresses the height of the target value g relative to the current value rᵤ^{(s)}. The health-oriented behavior tendency calculation unit 104 calculates the target difficulty level according to the following Equation (3).

$$[ Math . 3 ]$$

$${r}^{( d )} = \vert \frac{g - {r}_{u}^{( s )}}{{r}_{u}^{( s )}} \vert \qquad ( 3 )$$

However, the calculation method is not limited to the above-mentioned method as long as it follows the definition of the target difficulty level. For example, the health-oriented behavior tendency calculation unit 104 may calculate the target difficulty level as follows.

$$[ Math . 4 ]$$

$${r}^{( d )} = \frac{{g}_{u}}{{r}_{u}^{( s )}}$$

Also, the degree of execution is a value that expresses the degree of actual behavior toward the target value until the target value is achieved during the target period. Here, as an example, the degree of execution includes the execution frequency and the execution strength. As long as it follows the definition of the degree of execution, the scale constituting the degree of execution is not limited thereto. The execution frequency refers to the frequency of behavior toward the target value, and the execution strength refers to the average change amount per behavior toward the target value. For example, assuming that the L-th log is a log output when the target value is achieved for the first time, the health-oriented behavior tendency calculation unit 104 sets a vector obtained by extracting elements from the preprocessed health behavior data vector y* to the L-th element as y^{p}. y^{P} is calculated by the following Equation (4).

$$[ Math . 5 ]$$

$$\begin{matrix}{y}^{( p )} & = {( {y}_{i}^{*} \vert ( g - {y}_{{i}^{*} < L - 1} ) ( g - {y}_{j < L - 1}^{*} ) > 0 , ( g - {y}_{i < L - 1}^{*} ) ( g - {y}_{L - 1}^{*} ) \leq 0 )}^{T} \\ & = {( {y}_{0}^{*} , {y}_{L - 1}^{*} )}^{T}\end{matrix} \qquad ( 4 )$$

In this case, the execution frequency r^{(p, f)} is calculated by the following Equation (5).

$$[ Math . 6 ]$$

$${r}^{( p , f )} = \frac{{\sum }_{i} {c}_{i}^{( p )}}{\vert {y}^{( p )} \vert} ( {c}_{i}^{( p )} = \{ \begin{matrix}1 , & ( g - {y}_{i - 1}^{( p )} ) ( g - {y}_{i}^{( p )} ) > 0 \\ 0 , & ( g - {y}_{i - 1}^{( p )} ) ( g - {y}_{i}^{( p )} ) \leq 0\end{matrix} ) \qquad ( 5 )$$

Also, the execution strength r^{(p, s)} is calculated by the following Equation (6).

$$[ Math . 7 ]$$

$${r}^{( p , s )} = \frac{{\sum }_{i} {c}_{i}^{( p )} \vert \frac{{y}_{i}^{( p )} - {y}_{i - 1}^{( p )}}{{y}_{i}^{( p )}} \vert}{{\sum }_{i} {c}_{i}^{( p )}} \qquad ( 6 )$$

However, the calculation method is not limited to the above-mentioned method as long as it follows the definition of the degree of execution. For example, the health-oriented behavior tendency calculation unit 104 may calculate the execution frequency as follows:

$$[ Math . 8 ]$$

$${r}^{( p , f )} = {\sum }_{i} {c}_{i}$$

and may calculate the execution strength as follows.

$$[ Math . 9 ]$$

$${r}^{( p , s )} = \frac{{\sum }_{i} {c}_{i} \vert {y}_{i}^{p} - {y}_{i - 1}^{p} \vert}{{\sum }_{i} {c}_{i}}$$

The degree of achievement is a value that expresses the degree of achievement of the set target value. Here, as an example, the degree of achievement includes the achievement frequency and the achievement speed. As long as it follows the definition of the degree of achievement, the scale constituting the degree of achievement is not limited thereto. The achievement frequency refers to the frequency at which a value obtained by achieving the target value is recorded during the target period. The achievement speed refers to the speed until the target value is achieved for the first time. The achievement frequency r^{(a, f)} is calculated by the following Equation (7).

$$[ Math . 10 ]$$

$${r}^{( a , f )} = \frac{{\sum }_{i} {c}_{i}^{( a )}}{\vert {y}^{*} \vert} ( {c}_{i}^{( a )} = \{ \begin{matrix}1 , & g \geq {y}_{i}^{*} \\ 0 , & g < {y}_{i}^{*}\end{matrix} ) \qquad ( 7 )$$

Also, assuming that the L-th log is the log output when the target value is achieved for the first time, the achievement speed r^{(a, v)} is calculated by the following Equation (8).

$$[ Math . 11 ]$$

$${r}^{( a , ν )} = \frac{\vert {y}_{L}^{*} - {y}_{0}^{*} \vert}{{t}_{L}^{*} - {t}_{0}^{*}} \qquad ( 8 )$$

However, the calculation method is not limited to the above-mentioned method as long as it follows the definition of the degree of achievement. For example, the health-oriented behavior tendency calculation unit 104 may calculate the achievement frequency and the achievement speed as follows.

$$[ Math . 12 ]$$

$${r}^{( a , f )} = {\sum }_{i} {c}_{i}^{( a )} , {r}^{( a , v )} = \frac{\vert {y}_{L}^{*} - {y}_{0}^{*} \vert}{L}$$

Also, the degree of retention is a value indicating the degree to which a state close to the target value is maintained during the target period. The health-oriented behavior tendency calculation unit 104 evaluates how much the target value g is expected from the frequency distribution of the elements of the health behavior data vector y*, and calculates the degree of retention. The health-oriented behavior tendency calculation unit 104 calculates the degree of retention r^{(f)} by a kernel density function based on the frequency distribution of y*.

The health-oriented behavior tendency calculation unit 104 assumes y₀*, . . . , Y_{N-1}* as samples obtained from an independent identical distribution having a probability density function f, and calculates a kernel density estimator of a kernel function K and a smoothing parameter h as shown in the following Equation (9).

$$[ Math . 13 ]$$

$${\hat{f}}_{h} ( x ) = \frac{1}{Nh} {\sum }_{i} K ( \frac{x - {y}_{i}^{*}}{h} ) = \frac{1}{2 \sqrt{π} Nh} {\sum }_{i} exp ( - \frac{{( x - {y}_{i}^{*} )}^{2}}{2} ) \qquad ( 9 )$$

Here, the health-oriented behavior tendency calculation unit 104 calculates the degree of retention r^{(f)} as shown in the following Equation (10).

$$\begin{matrix}[ Math . 14 ] & \\ {r}^{( f )} = {\hat{f}}_{h} ( g ) & ( 10 )\end{matrix}$$

Note that the kernel function is set as follows:

$$K ( x ) = \frac{1}{2 \sqrt{π}} \cdot exp ( - \frac{{x}^{2}}{2} ) \qquad [ Math . 15 ]$$

but other settings may be used. An appropriate value is set for the smoothing parameter h. Also, the calculation method is not limited to the above-mentioned method as long as it follows the definition of the degree of retention.

The health-oriented behavior tendency calculation unit 104 outputs the calculated current value r^{(s)}, the target difficulty level r^{(d)}, the degree of execution r^{(p)}, the degree of achievement r^{(a)}, and the degree of retention r^{(f)} to the health-oriented behavior estimation model learning unit 109.

Next, the process of step S120 of the learning processing by the human characteristic data preprocessing unit 106 of the learning apparatus 10 will be described. The process to be described later is similar to the process of step S200 of the prediction processing by the human characteristic data preprocessing unit 201 of the prediction apparatus 20.

The human characteristic data preprocessing unit 106 converts the answer values in accordance with the nature of the scales of the answers to the questions of the questionnaire survey. Answer scales include a nominal scale, an ordinal scale, an interval scale, and a ratio scale, each of which is converted as follows.

The nominal scale is a scale used to distinguish attributes and categories. In the example of the human characteristic data 905 illustrated in FIG. 9, the answer values to the gender (Q1_1) and the marriage status (Q1_4) correspond to these values. The human characteristic data preprocessing unit 106 converts an answer value corresponding to the nominal scale in association with a value preset in the learning apparatus 10.

For example, the human characteristic data preprocessing unit 106 converts “female” into “0” and “male” into “1.” Note that the conversion method is not limited to the above-mentioned method as long as the attribute and category corresponding to the answer value can be distinguished even after conversion.

The ordinal scale is a scale which has meaning in the magnitude relation but does not have meaning in the difference or ratio. In the example of the human characteristic data 905 illustrated in FIG. 9, the answer values to the psychological characteristics (Q2_1 and Q2_2) and the health habits (Q4_1 and Q4_2) correspond to these values. The human characteristic data preprocessing unit 106 converts answer values corresponding to the ordinal scale so that the order relationship is maintained between the answer values.

For example, the human characteristic data preprocessing unit 106 converts “not at all” into 1, “no” into 2, “neither” into 3, “yes” into 4, and “very much” into 5. Note that the conversion method is not limited to the above-mentioned method as long as the order relationship of the answer values is maintained even after the conversion.

Interval scales and ratio scales are called quantitative data, and are scales in which intervals are set equally. In an example of the human characteristic data 905 illustrated in FIG. 9, the answer values relating to the age (Q1_2), the number of housemates (Q1_5), the cognitive characteristics (Q3_1, Q3_2), and the work productivity (Q5_1, Q5_2) correspond to interval scales or ratio scales. The human characteristic data preprocessing unit 106 does not convert these answer values.

Next, the process of step S130 of the learning processing by the human characteristic scale calculation unit 107 will be described. The process to be described later is similar to the process of step S201 of the prediction processing by the human characteristic scale calculation unit 202 of the prediction apparatus 20.

The human characteristic scale calculation unit 107 calculates a human characteristic scale of the learning object by using the converted answer values obtained by the human characteristic data preprocessing unit 106. The calculation method follows a method predetermined in the learning apparatus 10.

For example, in the example illustrated in FIG. 11, the human characteristic scale calculation unit 107 calculates a human characteristic scale called “Extraversion” by averaging the answer values of Q2_1* and Q2_2* in the preprocessed human characteristic data.

For example, when it is assumed that the human characteristic scale of M items is calculated, the human characteristic scale calculation unit 107 outputs a human characteristic scale vector x=(xᵢ|i=1, . . . , M)^{T} for each user, where xᵢ is the human characteristic scale of the item i.

The process of step S140 of the learning processing by the health-oriented behavior estimation model construction unit 108 will be described. The health-oriented behavior estimation model aims to predict the health-oriented behavior tendency from the human characteristic scale, and can be constructed by using any method as long as it follows a supervised learning method. Here, linear regression is used as an example. Linear regression is generally described as in the following Equation (11), where y is the objective variable, xᵢ is the explanatory variable, βᵢ is the coefficient of xᵢ, α is the constant term, and ε is the error term.

$$\begin{matrix}[ Math . 16 ] & \\ y = α + {β}_{1} {x}_{1} + … + {β}_{M} {x}_{M} + ε & ( 11 )\end{matrix}$$

Here, assuming that the parameter vector is ß=(α, β₁, . . . , β_{M})^{T} and the explanatory variable vector is x=(1, x₁, . . . , X_{M})^{T}, it is described as in the following Equation (12).

$$\begin{matrix}[ Math . 17 ] & \\ y = {x}^{T} β + ε & ( 12 )\end{matrix}$$

Here, the health-oriented behavior tendency r^{h} (h=(s), (d), (p, f), (p, s), (a, f), (a, v), (f)) of the user u is denoted by rᵤ^{h}. The vector composed of the human characteristic scale X_{u,i} possessed by the user u is denoted by Xᵤ=(1, X_{u, 1}, . . . , X_{u, M})^{T}. The constant term in the regression of the health-oriented behavior tendency r^{h} is denoted by α^{h}, the coefficient of the human characteristic scale of the item i is denoted by βᵢ^{h}, and the parameter vector having them as elements is denoted by β^{h}=(α^{h}, β₁^{h}, . . . , B_{M}^{h})^{T}. The error term of the user U in the regression of the health-oriented behavior tendency r^{h} is denoted by εᵤ^{h}. These variables are described as in Equation (13) based on Equation (12).

$$\begin{matrix}[ Math . 18 ] & \\ {r}_{u}^{h} = {x}_{u}^{T} {β}^{h} + {ε}_{u}^{h} & ( 13 )\end{matrix}$$

$$Here ,$$

$${r}^{h} = {( {r}_{1}^{h} , … , {r}_{N}^{h} )}^{T} , \qquad [ Math . 19 ]$$

$$X = {( {x}_{1} , … {x}_{N} )}^{T} = ( \begin{matrix}1 & {x}_{1 , 1} & … & {x}_{1 , M} \\ ⋮ & ⋮ & ⋱ & ⋮ \\ 1 & {x}_{N , 0} & … & {x}_{N , M}\end{matrix} ) ,$$

$${ε}^{h} = {( {ε}_{1}^{h} , … , {ε}_{N}^{h} )}^{T}$$

when the above equations are satisfied, Equation (13) is described as in Equation (14).

$$\begin{matrix}[ Math . 20 ] & \\ {r}^{h} = X {β}^{h} + {ε}^{h} & ( 14 )\end{matrix}$$

The health-oriented behavior estimation model construction unit 108 constructs the health-oriented behavior estimation model as the following Equation (15).

$$\begin{matrix}[ Math . 21 ] & \\ f ( X ) = X {β}^{h} & ( 15 )\end{matrix}$$

Next, the process of step S150 of the learning processing by the health-oriented behavior estimation model learning unit 109 will be described. The health-oriented behavior estimation model learning unit 109 receives the health-oriented behavior tendency r^{h} (h=(s), (d), (p, f), (p, s), (a, f), (a, v), (f)) from the health-oriented behavior tendency calculation unit 104, the human characteristic scale vector x from the human characteristic scale calculation unit 107, and f (X) from the health-oriented behavior estimation model construction unit 108, and learns the parameter β^{h}. Hereinafter, the learning procedure will be described.

The health-oriented behavior estimation model learning unit 109 standardizes the human characteristic scale x_{u,i} of the item i of the user u according to the following Equation (16).

$$\begin{matrix}[ Math . 22 ] & \\ {z}_{u , i} = \frac{{x}_{u , i} - {μ}_{i}}{{σ}_{i}} & ( 16 )\end{matrix}$$

Here, μᵢ and σᵢ are the average value and variance of the human characteristic scale xᵢ of the item i for the user, respectively.

Next, the health-oriented behavior estimation model learning unit 109 obtains a parameter for minimizing an error between an estimation result of the health-oriented behavior estimation model and a true value. An example of calculating parameters by the least squares method will be shown. The health-oriented behavior estimation model learning unit 109 sets Z to a standardized human characteristic matrix in which an element x_{u,i} of a matrix X which is an input variable of the health-oriented behavior estimation model is replaced with Z_{u,i}, and constructs Z as shown in the following Equation (17).

$$\begin{matrix}[ Math . 23 ] & \\ Z = ( \begin{matrix}1 & {z}_{1 , 1} & … & {z}_{1 , M} \\ ⋮ & ⋮ & ⋱ & ⋮ \\ 1 & {z}_{N , 1} & … & {z}_{N , M}\end{matrix} ) & ( 17 )\end{matrix}$$

Assuming that the health-oriented behavior tendency vector estimated from the standardized human characteristic matrix Z is \hat{ }r^{h}, the estimated error vector ε^{h} is described as in the following Equation (18).

$$\begin{matrix}[ Math . 24 ] & \\ \begin{matrix}{ε}^{h} = {r}^{h} - {\hat{r}}^{h} \\ = {r}^{h} - f ( Z ) \\ = {r}^{h} - Z {β}^{h}\end{matrix} & ( 18 )\end{matrix}$$

The health-oriented behavior estimation model learning unit 109 obtains a parameter β^{h} for minimizing the error vector ε^{h} by solving an optimization problem such as the following Equation (19).

$$\begin{matrix}[ Math . 25 ] & \\ {arg min}_{{β}^{h}} {( {ε}^{h} )}^{T} {ε}^{h} = {arg min}_{{β}^{h}} {( {r}^{h} - Z {β}^{h} )}^{T} ( {r}^{h} - Z {β}^{h} ) & ( 19 )\end{matrix}$$

Specifically, the health-oriented behavior estimation model learning unit 109 sets a loss function L (β^{h})=(r^{h}−Zβ^{h})^{T} (r^{h}−Zβ^{h}), and searches for a point where a gradient of the loss function β^{h} becomes 0. Therefore, the parameter β^{h} for the health-oriented behavior tendency h is learned as in the following Equation (20).

$$\begin{matrix}[ Math . 26 ] & \\ \frac{\partial L ( {β}^{h} )}{{β}^{h}} = 0 ⇔ - 2 {Z}^{T} {r}^{h} + 2 {Z}^{T} Z {β}^{h} = 0 ⇔ {β}^{h} = {( {Z}^{T} Z )}^{- 1} {Z}^{T} {r}^{h} & ( 20 )\end{matrix}$$

Although an example of learning parameters using the least squares method has been described, the method of learning parameters is not limited to the above-mentioned method as long as it is a method of obtaining parameters for minimizing the error between the estimation result of the health-oriented behavior estimation model and the true value.

The health-oriented behavior estimation model learning unit 109 substitutes the obtained parameter β^{h} into a health-oriented behavior estimation model f, and stores the parameter-substituted health-oriented behavior estimation model in the health-oriented behavior estimation model storage unit 110.

Next, the process of step S202 of the prediction processing by the health-oriented behavior prediction unit 204 will be described. It is assumed that a linear regression model f(X) is stored in the health-oriented behavior estimation model storage unit 203 as the health-oriented behavior estimation model. When the human characteristic scale x′_{u,i} and the health-oriented behavior estimation model f(X) are acquired, the health-oriented behavior prediction unit 204 converts the human characteristic scale x′_{u,i} as shown in the following Equation (21).

$$\begin{matrix}[ Math . 27 ] & \\ {z}_{u , i}^{′} = \frac{{x}_{u , i}^{′} - {μ}_{i}^{′}}{{σ}_{i}^{′}} & ( 21 )\end{matrix}$$

Here, μ'ᵢ and σ's are the average value and variance of the human characteristic scale for each user, respectively. Here, a matrix having z′_{u,i} as elements is represented by Equation (22).

$$\begin{matrix}[ Math . 28 ] & \\ {Z}^{′} = ( \begin{matrix}1 & {z}_{1 , 1}^{′} & … & {z}_{1 , M}^{′} \\ ⋮ & ⋮ & ⋱ & ⋮ \\ 1 & {z}_{N , 1}^{′} & … & {z}_{N , M}^{′}\end{matrix} ) & ( 22 )\end{matrix}$$

The estimation result \hat{ }r^{h}=(\hat{ }r^{h}|u=1, . . . , N)^{T} of the health-oriented behavior is obtained from the health-oriented behavior estimation model f(X) and the learned parameter vector β^{h}=(Z^{T}Z)⁻¹Z^{T}r^{h} as shown in the following Equation (23).

$$\begin{matrix}[ Math . 29 ] & \\ {\hat{r}}^{h} = f ( {Z}^{′} ) = {Z}^{′} {β}^{h} = {{Z}^{′} ( {Z}^{T} Z )}^{- 1} {Z}^{T} {r}^{h} & ( 23 )\end{matrix}$$

The health-oriented behavior prediction unit 204 outputs the estimation result \hat{ }r^{h} of the health-oriented behavior.

### Example of Hardware Configuration According to Present Embodiment

The learning apparatus 10 and the prediction apparatus 20 can be implemented, for example, by causing a computer to execute a program describing the processing details described in the present embodiment. Note that this “computer” may be a physical machine or a virtual machine on the cloud. When using a virtual machine, the “hardware” described here is virtual hardware.

The above program can be stored and distributed by being recorded in a computer-readable recording medium (a portable memory or the like). Furthermore, the above program can also be provided through a network such as the Internet or an electronic mail.

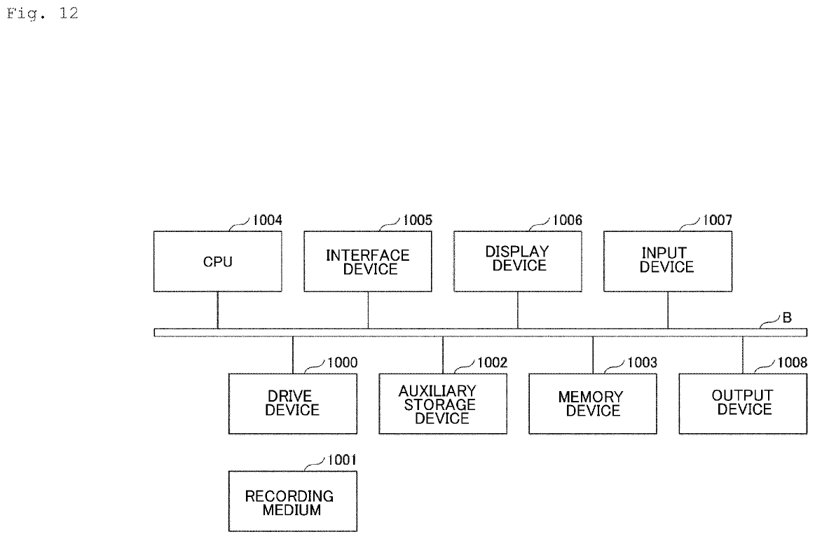

FIG. 12 is a diagram illustrating an example of a hardware configuration of the above computer. The computer illustrated in FIG. 12 includes a drive device 1000, an auxiliary storage device 1002, a memory device 1003, a CPU 1004, an interface device 1005, a display device 1006, an input device 1007, an output device 1008, and the like, which are connected to each other via a bus B.

The program for implementing the processing in the computer is provided by, for example, a recording medium 1001 such as a CD-ROM or a memory card. When the recording medium 1001 in which the program is stored is set in the drive device 1000, the program is installed from the recording medium 1001 to the auxiliary storage device 1002 through the drive device 1000. However, the program need not necessarily be installed from the recording medium 1001, and may be downloaded from another computer via a network. The auxiliary storage device 1002 stores the installed program and stores necessary files, data, and the like.

The memory device 1003 reads the program from the auxiliary storage device 1002 and stores the program in response to an instruction to start the program. The CPU 1004 achieves functions related to the device according to the program stored in the memory device 1003. The interface device 1005 is used as an interface for connection to a network. The display device 1006 displays a graphical user interface (GUI) or the like according to the program. The input device 1007 includes a keyboard and mouse, buttons, a touch panel, or the like, and is used to input various operation instructions. The output device 1008 outputs a calculation result. The computer may include a graphics processing unit (GPU) or tensor processing unit (TPU) instead of the CPU 1004, or may include a GPU or TPU in addition to the CPU 1004. In this case, the processing may be shared and executed such that the GPU or TPU executes processing requiring special operation such as a neural network, and the CPU 1004 executes other processing.

### Effect of Present Embodiment

The learning apparatus 10 according to the present embodiment performs learning considering not only basic items such as gender, age, and BMI, but also various human characteristics such as psychological characteristics, cognitive characteristics, health habits, and work productivity. Thus, the estimation accuracy of the model for estimating the health-oriented behavior tendencies can be improved. Further, the prediction apparatus 20 performs prediction considering not only basic items such as gender, age, and BMI, but also various human characteristics such as psychological characteristics, cognitive characteristics, health habits, and work productivity. Thus, the prediction accuracy of health-oriented behavior tendencies can be improved.

Further, the learning apparatus 10 according to the present embodiment calculates various values indicating the health-oriented behavior tendencies including the degree of retention or the like on the basis of the health behavior data and the health target data, and learns the parameters of the health-oriented behavior estimation model by machine learning based on the calculated values. By evaluating various values indicating the health-oriented behavior tendencies including the degree of retention or the like, the health-oriented behavior can be evaluated from a new viewpoint which has not been evaluated in the past. This can evaluate the health-oriented behavior tendencies from multiple angles and further improve the prediction accuracy.

### Summary of Embodiment

The present specification describes at least the prediction apparatus, the learning apparatus, the prediction method, the learning method, and the program described in each of the following items.

### Item 1

A prediction apparatus for predicting a health-oriented behavior tendency of a prediction object, the prediction apparatus comprising:

a health-oriented behavior estimation model storage unit that stores a health-oriented behavior estimation model for estimating a health-oriented behavior based on a relationship between a health-oriented behavior and human characteristics;

a human characteristic scale calculation unit that calculates a plurality of human characteristic scales based on human characteristic data indicating human characteristics of a prediction object; and a health-oriented behavior prediction unit that predicts a health-oriented behavior tendency based on the plurality of calculated human characteristic scales by using the health-oriented behavior estimation model.

### Item 2

The prediction apparatus according to Item 1, further comprising a human characteristic data preprocessing unit that converts the human characteristic data into a predetermined format in accordance with a nature of a scale of an answer value to a question item included in the human characteristic data, wherein the human characteristic scale calculation unit calculates the plurality of human characteristic scales based on the human characteristic data converted into the format.

### Item 3

The prediction apparatus according to Item 2, wherein the human characteristic scale calculation unit converts the format of the human characteristic data according to any one of a nominal scale, an ordinal scale, an interval scale, and a ratio scale as the scale of the answer value.

### Item 4

A learning apparatus for learning a parameter of a health-oriented behavior estimation model for estimating a health-oriented behavior, the learning apparatus comprising:

a health-oriented behavior estimation model storage unit that stores a health-oriented behavior estimation model for estimating a health-oriented behavior based on a relationship between a health-oriented behavior and human characteristics; a health-oriented behavior tendency calculation unit that calculates a value indicating a health-oriented behavior tendency of a learning object based on health target data and health behavior data of the learning object;

a human characteristic scale calculation unit that calculates a plurality of human characteristic scales based on human characteristic data indicating human characteristics of the learning object; and a health-oriented behavior estimation model learning unit that learns a parameter of the health-oriented behavior estimation model based on the plurality of human characteristic scales and the value indicating the health-oriented behavior tendency.

### Item 5

The learning apparatus according to Item 4, wherein the health-oriented behavior tendency calculation unit calculates values indicating a plurality of health-oriented behavior tendencies including a degree of retention indicating a degree to which a state close to a target value is maintained during a target period.

### Item 6

A prediction method executed by a prediction apparatus that stores a health-oriented behavior estimation model for estimating a health-oriented behavior based on a relationship between a health-oriented behavior and human characteristics, the prediction method comprising:
a step of calculating a plurality of human characteristic scales based on human characteristic data indicating human characteristics of a prediction object; and
a step of predicting a health-oriented behavior tendency based on the plurality of calculated human characteristic scales by using the health-oriented behavior estimation model.

### Item 7

A learning method executed by a learning apparatus that stores a health-oriented behavior estimation model for estimating a health-oriented behavior based on a relationship between a health-oriented behavior and human characteristics, the learning method comprising:
a step of calculating a value indicating a health-oriented behavior tendency of a learning object based on health target data and health behavior data of the learning object;
a step of calculating a plurality of human characteristic scales based on human characteristic data indicating human characteristics of the learning object; and
a step of learning a parameter of the health-oriented behavior estimation model based on the plurality of human characteristic scales and the value indicating the health-oriented behavior tendency.

### Item 8

A program for causing a computer to function as each unit in the prediction apparatus according to any one of Items 1 to 3, or a program for causing a computer to function as each unit in the learning apparatus according to Item 4 or 5.

Although the present embodiment has been described above, the present invention is not limited to such a specific embodiment, and various modifications and changes can be made within the scope of the gist of the present invention described in the claims.

### REFERENCE SIGNS LIST

10 learning apparatus
20 prediction apparatus
101 health target data storage unit
102 health behavior data storage unit
103 health behavior data preprocessing unit
104 health-oriented behavior tendency calculation unit
105 human characteristic data storage unit
106 human characteristic data preprocessing unit
107 human characteristic scale calculation unit
108 health-oriented behavior estimation model construction unit
109 health-oriented behavior estimation model learning unit
110 health-oriented behavior estimation model storage unit
201 human characteristic data preprocessing unit
202 human characteristic scale calculation unit
203 health-oriented behavior estimation model storage unit
204 health-oriented behavior prediction unit
1000 drive device
1001 recording medium
1002 auxiliary storage device
1003 memory device
1004 CPU
1005 interface device
1006 display device
1007 input device
1008 output device

1. A prediction apparatus for predicting a health-oriented behavior tendency of a prediction object, the prediction apparatus comprising:
a health-oriented behavior estimation model storage unit that stores a health-oriented behavior estimation model for estimating a health-oriented behavior based on a relationship between a health-oriented behavior and human characteristics;
a processor; and
a memory storing program instructions that cause the processor to:
calculate a plurality of human characteristic scales based on human characteristic data indicating human characteristics of a prediction object; and
predict a health-oriented behavior tendency based on the plurality of calculated human characteristic scales by using the health-oriented behavior estimation model.

2. The prediction apparatus according to claim 1, wherein the program instructions further cause the processor to convert the human characteristic data into a predetermined format in accordance with a nature of a scale of an answer value to a question item included in the human characteristic data,
wherein the processor calculates the plurality of human characteristic scales based on the human characteristic data converted into the format.

3. The prediction apparatus according to claim 2, wherein the processor converts a format of the human characteristic data according to any one of a nominal scale, an ordinal scale, an interval scale, and a ratio scale as the scale of the answer value.

4. A learning apparatus for learning a parameter of a health-oriented behavior estimation model for estimating a health-oriented behavior, the learning apparatus comprising:
a processor; and
a memory storing program instructions that cause the processor to:
store a health-oriented behavior estimation model for estimating a health-oriented behavior based on a relationship between a health-oriented behavior and human characteristics;
calculate a value indicating a health-oriented behavior tendency of a learning object based on health target data and health behavior data of the learning object;
calculate a plurality of human characteristic scales based on human characteristic data indicating human characteristics of the learning object; and
learn a parameter of the health-oriented behavior estimation model based on the plurality of human characteristic scales and the value indicating the health-oriented behavior tendency.

5. The learning apparatus according to claim 4, wherein the processor calculates values indicating a plurality of health-oriented behavior tendencies including a degree of retention indicating a degree to which a state close to a target value is maintained during a target period.

6. A prediction method executed by a prediction apparatus that stores a health-oriented behavior estimation model for estimating a health-oriented behavior based on a relationship between a health-oriented behavior and human characteristics, the prediction method comprising:
calculating a plurality of human characteristic scales based on human characteristic data indicating human characteristics of a prediction object; and
predicting a health-oriented behavior tendency based on the plurality of calculated human characteristic scales by using the health-oriented behavior estimation model.

7. A learning method executed by a learning apparatus that stores a health-oriented behavior estimation model for estimating a health-oriented behavior based on a relationship between a health-oriented behavior and human characteristics, the learning method comprising:
calculating a value indicating a health-oriented behavior tendency of a learning object based on health target data and health behavior data of the learning object;
calculating a plurality of human characteristic scales based on human characteristic data indicating human characteristics of the learning object; and
learning a parameter of the health-oriented behavior estimation model based on the plurality of human characteristic scales and the value indicating the health-oriented behavior tendency.

8. A non-transitory computer-readable recording medium having stored therein a program for causing a computer to execute the prediction method according to claim 6.

9. A non-transitory computer-readable recording medium having stored therein a program for causing a computer to execute the learning method according to claim 7.
